(19) United States
(12) Patent Application Publication
(10) Pub. No.: US 2009/0082731 A1
(43) Pub. Date: Mar. 26, 2009

(54) DILATING TROCAR CANNULA

(75) Inventor: Victor Carlos Moreno, Terrace Park, OH (US)

Correspondence Address: Ethicon Endo-Surgery/Nutter, McClennen & Fish LLP, World Trade Center West, 155 Seaport Blvd., Boston, MA, 02210-2604, US

(73) Assignee: ETHICON ENDO-SURGERY, INC., Cincinnati, OH (US)

(21) Appl. No.: 11/858,490

(22) Filed: Sep. 20, 2007

(51) Int. Cl. A61M 25/06; A61M 25/00

(52) U.S. Cl. 604/158; 604/527

(57) ABSTRACT

Various methods and devices are provided for dilating tissue. In one embodiment, a braided tube is provided having an inner lumen extending along a longitudinal axis between first and second ends, and at least one axial support member slidably disposed along a length of the braided tube. In use, the axial support member(s) can move longitudinally to selectively increase and decrease a diameter of the inner lumen of the braided tube, and thereby dilate tissue.

### FIELD

The present application relates to methods and devices for dilating tissue, and more specifically to a braided tube adapted to dilate tissue.

### BACKGROUND

Access ports are widely used in medical procedures to gain access to anatomical cavities ranging in size from the abdomen to small blood vessels, such as veins and arteries, epidural, pleural and subarachnoid spaces, heart ventricles, and spinal and synovial cavities. The use of access ports has become more common as they provide minimally invasive techniques for establishing a portal for a number of procedures, such as those involving the abdominal cavity.

A trocar is one type of access post that is commonly used to provide a minimally invasive pathway for accessing a surgical site. Trocars generally include a cutting assembly (or obturator) that is disposed within an outer cannula. The sharp distal end of the cutting assembly, with the cannula disposed therearound, is urged through the skin until it enters the anatomical cavity being penetrated. The cutting assembly is then withdrawn from the cannula, which remains in place to provide a passageway through which access to the anatomical cavity is provided for other surgical devices.

While effective, there can be many disadvantages when using a typical trocar assembly. For example, the size of the access port is related to the size of the cut made through the skin. Therefore, if a large opening is needed for access to a body cavity, a large wound will need to be created. Additionally, the trocar could extend a distance above the skin and surface through which it is inserted depending on the length of the trocar, which could interfere with access to the surgical field.

Accordingly, there is a need for improved methods and devices for providing access through tissue to a surgical site, and in particular for providing minimally invasive means for dilating the tissue surrounding the access site.

### SUMMARY

The present invention provides various methods and devices for dilating tissue. In one embodiment, a trocar cannula is provided and includes a braided tube having an inner lumen extending along a longitudinal axis between first and second ends, and at least one axial support member slidably disposed along a length of the braided tube and adapted to move longitudinally to selectively increase and decrease a diameter of the inner lumen of the braided tube. The axial support member(s) can be coupled to an actuator adapted to slide the axial support member(s) longitudinally along the cannula. The axial support member(s) can have a proximal end coupled to the actuator and a distal end coupled to a distal end of the braided tube. The actuator can be adapted to pull the axial support member(s) proximally to increase the diameter of the inner lumen and decrease a length of the braided tube.

The axial support member(s) can have a variety of configurations and be coupled to the braided tube in a number of ways. In one embodiment, the axial support member(s) can be slidably mated to the braided tube along the entire length thereof between the proximal and distal ends, or along only a portion thereof, such as between the distal end of the braided tube to a position distal to a proximal end of the braided tube. The axial support member(s) can also be woven into the braided tube, or mated to an inner or outer portion thereof. In another embodiment, the trocar cannula can include a plurality of axial support members spaced radially around the braided tube. The trocar cannula can also optionally include one or more protrusions extending radially outward from the braided tube and adapted to engage the tissue.

In another exemplary embodiment, the braided tube can be configured to be substantially impermeable to fluid. This can be achieved in a variety of ways. For example, a flexible sheath can be disposed around an inner or outer sidewall of the braided tube such that the sidewall is substantially fluid impermeable, or the braided tube can include a coating formed thereon such that the braided tube is fluid impermeable.

In another embodiment, a trocar is provided having an obturator and a cannula removably disposable within the obturator. The cannula can be formed from a braided tube having at least one axial support member slidably disposed along a length thereof and adapted to move to vary a diameter of the cannula. In one embodiment, the cannula can include four axial support members spaced radially around the cannula. The trocar can also include an actuator coupled to the axial support member(s) and adapted to slide the axial support member(s) along a length of the cannula. In an exemplary embodiment, the axial support member(s) include a proximal end coupled to the actuator and a distal end coupled to a distal end of the cannula.

In certain aspects, the actuator can be adapted to rotate about an axis of the cannula to slide the axial support member(s) along the axis. For example, the actuator can be in the form of a rotatable knob adapted to rotate to gather the axial support member(s) around a spool rotatably coupled to the rotatable knob, thereby pulling on the axial support member(s) along the axis. A locking mechanism, such as a ratchet, can be coupled to the rotatable knob for maintaining the rotatable knob in a fixed position, thereby maintaining the cannula in a fixed dilated position. The locking mechanism can also include a release mechanism adapted to release the axial support member(s) to decrease the diameter of the cannula.

Methods for dilating tissue are also provided, and in one embodiment the method can include inserting a cannula into tissue and pulling at least one axial support member slidably disposed along a length of the cannula to decease a length of the cannula and increase a diameter of the cannula and thereby dilate the tissue. The cannula can be inserted into tissue with an obturator disposed therein, or using other techniques known in the art. In one embodiment, pulling the axial support member(s) can include rotating a rotatable knob coupled to the axial support member(s) such that the axial support member(s) is gathered around a spool. A ratchet or other locking mechanism can maintain the rotatable knob in a fixed position to thereby maintain the axial support member(s) in a desired gathered position around the spool, thereby maintaining the cannula at a desired increased diameter. The method can also include releasing a release mechanism associated with the rotatable knob to release the axial support member(s) from the spool and decrease the diameter of the cannula.

### BRIEF DESCRIPTION OF THE DRAWINGS

The invention will be more fully understood from the following detailed description taken in conjunction with the accompanying drawings, in which:

FIG. 1 is a perspective view of a trocar cannula assembly including an obturator disposed within a cannula formed from a braided tube;

FIG. 2 is a perspective view of the braided tube of FIG. 1;

FIG. 3 is a side view of the braided tube of FIG. 2 in a first, undilated configuration;

FIG. 4 is a side view of the braided tube of FIG. 2 in a second, dilated configuration;

FIG. 5 is a perspective view of an actuator used to move the braided tube between the first, undilated configuration and the second, dilated configuration;

FIG. 6 is a side view of the trocar cannula assembly of FIG. 1 after the trocar cannula assembly has been inserted through the abdominal wall;

FIG. 7 is a side view of the braided tube of FIG. 6 inserted through the abdominal wall in the first, undilated configuration; and

FIG. 8 is a side view of the braided tube of FIG. 6 inserted through the abdominal wall in the second, dilated configuration.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for dilating tissue using a braided tube. In general, the braided tube is configured to move between a first, undilated configuration, and a second, dilated configuration. As the braided tube moves to the second configuration, the length of the braided tube decreases and the diameter of the braided tube increases. This allows the braided tube to dilate tissue when the braided tube is disposed through tissue, thereby forming an enlarged passageway through the tissue.

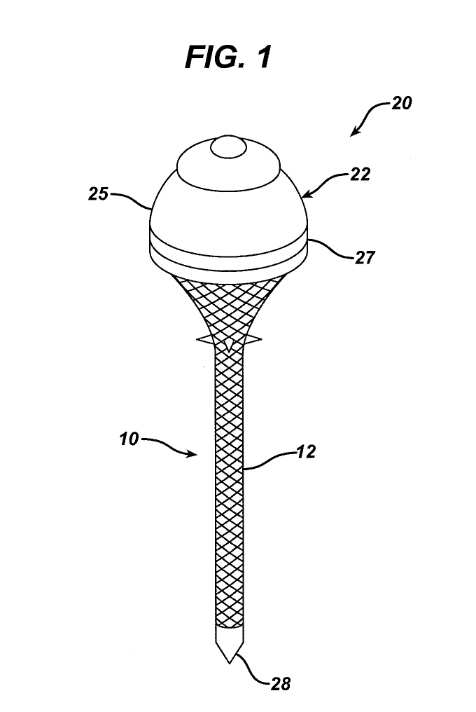

FIG. 1 illustrates one exemplary embodiment of a braided tube 12 used to form a cannula 10 for use with a trocar assembly 20. The trocar assembly 20 generally includes an obturator 22 that is slidably disposed through the cannula 10, and that has a distal end that extends beyond the distal end of the braided tube 12 for penetrating through tissue. Once inserted, the obturator 22 can be removed from the cannula 10 so that the cannula 10 provides a working channel through the tissue for inserting various instruments. The size of the working channel can be adapted as necessary by dilating the braided tube 12 of the cannula 10. A person skilled in art will appreciate that, while the braided tube 12 is shown in connection with a trocar assembly, the braided tube 12 can be incorporated into virtually any device in which it is necessary to dilate tissue.

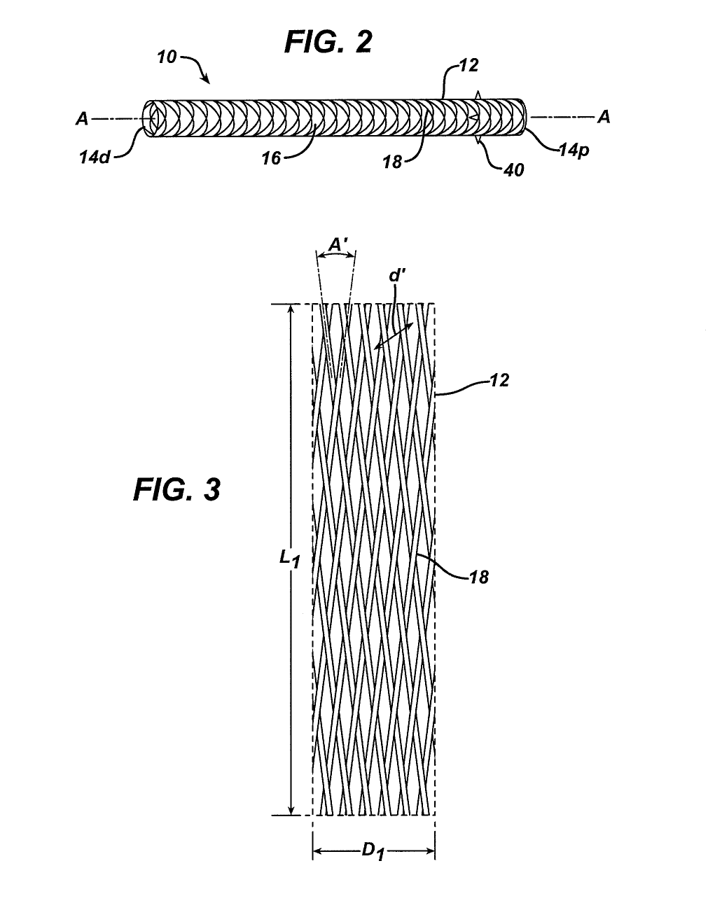

FIG. 2 illustrates the braided tube 12 of the cannula 10 in more detail. As shown, the braided tube 12 includes an inner lumen extending along a longitudinal axis A between a proximal end 14p and a distal end 14d thereof. The braided tube 12 can be formed in a variety of ways, but in one embodiment it can be formed by interweaving a plurality of filaments 18 around a central mandrel via a braiding machine. The interwoven braided filaments 18 can be spaced so as to allow the filaments 18 to move relative to each other. The term “filament” as used herein is broadly defined to cover any element with an elongated configuration, including but not limited to a thread, fiber, cord, string, yarn, twine, rope, line, cable, wire, ribbon, tape, or the like. The filaments 18 in the braided tube 12 may all be the same type and material, or a composite of different of types or materials. By way of non-limiting example, a maypole type of braiding machine, as sold by Steeger USA, Inc. of Spartanburg, S.C., or by the New England Butt Division of Wardwell Braiding Machine Company, can be used in construction of the braided tube 12. A person of ordinary skill in the art will appreciate that braiding machines of this type use two groups of carriers, where each carrier carries a spool of the filament 18 to be presented by the respective carrier. Carriers are arranged in a circular array around a braiding axis of the central mandrel and are driven in one direction about that axis. Carriers of the first group are arrayed around the braiding axis and are driven in clockwise direction for example. Carriers of the second group are also arrayed, in a circular array around the braiding axis, in an alternating order with respect to carriers of the first group, and are driven in the opposite direction about the braiding axis. As the carriers move, the filaments 18 are pulled off their respective spools and laid out onto the central mandrel forming the braided tube 12. A person skilled in the art will also appreciate any type of machine capable of forming the braided tube 12 can be used in place of a maypole type braiding machine described above.

The filaments 18 used to make the braided tube 12 can also be formed from a variety of materials. By way of non-limiting example, the filaments 18 can be formed from polyester, cotton, polyamide, polyalkane, polyurethane, PET, PBT, nylon, PEEK, PE, glass fibre, metal wire, acrylic materials, and the like, or any composition of the mentioned materials. They may be in the form a monofilament or a multifilament and may have a cross section in the shape of any geometrical form, such as a cross section in the form of a rectangle, square or a circle. For instance, in case of a circle they may have a diameter range of about 0.005 to about 0.04 inches. A person skilled in the art will appreciate that the porosity of the braided tube 12 and the size of the windows (i.e., the spaces between the interlaced filaments 18) formed by the intersecting filaments 18 are a function of the perpendicular distance (d) between the parallel filaments, the thickness of the filaments, and the intersection angle (A). The arrangement of the filament braiding will change the characteristics of the braided flexible tube. For example, the porosity of the braided tube 12 influences its flexibility and its hoop strength. For instance, if all other variables are held constant, the lower the porosity of a braided tube then the higher its hoop strength becomes. Decreasing the initial intersection angle (A), increasing the thickness of the filaments or their numbers, and decreasing the perpendicular distance between the parallel filaments are all ways to reduce the porosity of the braided flexible tube. In one embodiment, the intersection angle (A) has an angle range of more than 0 degrees and less than 180 degrees. In another embodiment, the intersection angle range between 10 and 170 degrees.

The particular shape and size of the braided tube 12 can also vary. In an exemplary embodiment, the braided tube 12 is cylindrical and has a diameter that is consistent along its length. However, in other embodiments, a portion of the braided tube 12 can be flared outward, such as a proximal portion of the braided tube 12, to facilitate attachment to a housing, actuator, and/or other device. The particular length and diameter will vary depending on the intended use. For example, where the braided tube 12 forms a trocar cannula 10, as in the illustrated embodiment, the braided tube 12 preferably a diameter in the first, undilated configuration that is sufficient to receive an elongate shaft of the obturator 22, and has a length that is slightly less then a length of the elongate shaft of the obturator 22 such that a tip 28 of the obturator 22 can extend distally beyond a distal end of the braided tube 12 to allow the tip 28 to come in contact with and penetrate tissue as the device is advanced. The size of the braided tube 12 can also vary depending on the tissue through which the braided tube 12 will be inserted. Preferably, the braided tube 12 has a length when in the second, dilated configuration that is substantially equal to or greater than a depth of tissue through which the braided tube 12 is inserted. This allows the distal end 14d of the braided tube 12 to be positioned just distal of an inner wall of the tissue through which the cannula 10 is inserted to prevent the braided tube 12 from extending too far into the cavity which the cannula 10 has penetrate. The proximal end 14p of the braided tube 12 can be just proximal of the outer wall of the tissue through which the cannula 10 has been inserted to provide a low profile. In an exemplary embodiment, the length is in the range of about 50 mm to 150 mm, and the diameter is in the range of about 5 mm to 25 mm.

The braided tube 12 can also include features to engage the tissue surrounding the braided tube 12 when the braided tube 12 is inserted through tissue. In one embodiment, the braided tube 12 can include one or more protrusions extending radially outward from the braided tube 12 and adapted to engage the tissue. The protrusions can have a variety of shapes and sizes. FIG. 2 illustrates teeth 40 positioned radially around a proximal portion of the braided tube 12 and having a tapered configuration with a sharpened tip for engaging tissue surrounding the braided tube 12. The protrusions can have a variety of other configurations adapted to engage tissue, including a flange formed around the braided tube 12. The protrusions can also be located at a variety of positions along the braided tube 12. In the illustrated embodiment, as indicated above, the protrusions are located just distal of the proximal end 14p of the braided tube 12 to engage tissue in the abdominal wall. A person skilled in the art will appreciate that any mechanism can be used to engage tissue surrounding the cannula 10, and that the cannula 10 can simply engage tissue using an interference fit.

The braided tube 12 can also include features to provided a barrier between the tissue and the braided tube 12 such that the sidewall of the braided tube 12 is substantially fluid impermeable. In one embodiment, the braided tube 12 can include a flexible sheath disposed around an inner or outer sidewall of the braided tube 12 that provides a substantially fluid impermeable barrier to prevent fluid from flowing through the sidewall of the braided tube 12. The flexible sheath can be made from a variety of materials, such as, for example, an elastomeric sheath. In another embodiment, the braided tube 12 can include a coating formed thereon to provide a substantially fluid impermeable barrier. The coating can be formed from a variety of materials, such as a polymer coating that provides a smooth outer and/or inner surface to the braided tube 12. For instance, the braided tube 12 may be dipped into a polymer solution. Polymers that may be used include, by way of non-limiting example, biocompatible polymers such as polyvinyl chloride, polyolefin (e.g., polyethylene, polypropylene, ethylene-vinylacetate copolymer), polyamide, polyester (e.g., polyethylene terephthalate (PET), polybutylene terephthalate), polyurethane, polystyrene resin, fluoro-based resin (e.g., polytetrafluoroethylene, ethylene-tetrafluoroethylene copolymer), polyimide, and the like; and various elastomers such as polyurethane-based elastomer, polyesterbased elastomer, polyolefin-based elastomer, polyamide-based elastomer, silicone rubber, latex rubber, and combinations thereof.

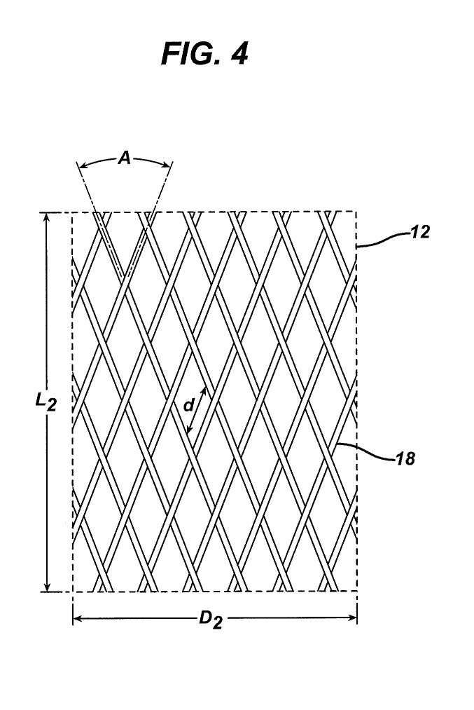

The braided tube 12 can also include one or more axial support members 16 disposed along a length thereof. While FIG. 2 illustrates only one axial support member 16, the braided tube 12 can include any number of axial support members 16 disposed therein in any configuration. For example, the braided tube 12 can include a plurality of axial support members 16 spaced circumferentially around the braided tube 12. The axial support member 16 can be adapted to move longitudinally along the length of the braided tube 12 to selectively increase and decrease the diameter of the braided tube 12. In particular, the axial support member 16 can move the braided tube 12 between a first, undilated configuration having a first diameter D₁ and a first length L₁, shown in FIG. 3, and a second, dilated position having a second diameter D₂ and a second length L₂, shown in FIG. 4, where D₁ is less than D₂ and L₁ is greater than L₂. Thus, as the braided tube 12 is dilated, the diameter of the braided tube 12 will increase and the length of the braided tube 12 will decrease. The axial support member 16 can have a variety of configurations, and it can be flexible or rigid depending on the intended use. In one exemplary embodiment, the axial support member 16 is semi-rigid to provide structural integrity to the braided tube 12. The axial support member 16 can also be disposed along the braided tube 12 in a variety of ways, including being woven into the braided tube 12. The axial support member 16 can further be coupled along certain portions of the braided tube 12 to facilitate movement of the braided tube 12 between the first and second configurations. For example, the axial support member 16 can only mate to a portion of the braided tube 12. For example, it can be in contact with the braided tube from its distal end 14d to a position distal to the proximal end 14p of the braided tube 12. The remaining proximal portion of the axial support member 16 can be separated form the braided tube 12 and can extend proximally inward or outward from the braided tube 12 to mate to an actuator, housing, or other structure, as will be discussed in more detail below. In an exemplary embodiment, at least the distal end of the axial support member 16 is mated to the distal end 14d of the braided tube 12. In use, the axial support member 16 can be pulled proximally to pull the distal end 14d of the braided tube 12 toward the proximal end 14p to thereby increase the diameter of the braided tube 12 and decrease the length of the braided tube 12. Distal movement of the axial support member 16 will allow the braided tube 12 to return to its first, undilated configuration.

FIGS. 3-4 depict the transition that may take place in the wall of the braided tube 12 when the braided tube 12 moves from the first, undilated configuration to the second, dilated configuration as the axial support member 16 is moved longitudinally along the length of the braided tube 12. Upon movement of the axial support member 16, the braid filaments 18 of the braided tube 12 will move relative to each other. An intersection angle A may drop to a smaller angle A′, which in turn may decrease the perpendicular distance between the parallel braid filaments 18 from a distance d to a distance d′ reducing the porosity of the wall of the braided tube 12 and decreasing its length from an initial length L₁ to a final length L₂. The reduction in porosity will yield an increase in the rigidity and hoop strength, which tends to lock it in a desired orientation.

In order for the axial support member 16 to move to vary the diameter of the cannula 10, the axial support member 16 can be coupled at its proximal end to an actuator formed on or mated to the cannula housing 27. The cannula housing 27 can have a variety of configurations, but it preferably includes a lumen formed therethrough and aligned with the lumen on the braided tube 12 to allow instruments to be passed through the cannula 10. A distal end of the housing 27 can be mated to the proximal end 14p of the braided tube 12. While not shown, the opening formed in the proximal-most end of the housing 27 of the cannula 10 can include a seal disposed therein and effective to engage an outer surface of various devices inserted into the cannula 10. The seal is particularly useful during insufflation as it can prevent gases from escaping through the assembly. In particular, the seal can permit the passage of the obturator 22 and/or various other surgical instruments through the cannula 10 while limiting or preventing the passage of fluid or gas therethrough. Due to the flexibility of the braided tube 12 adjacent the housing, the seal can have a fixed position within the housing, as the braided tube 12 will allow the housing 27 is pivot relative to the braided tube 12. This is particularly advantageous as it allows devices to be inserted through the cannula 10 at various insertion angles without breaking the seal formed around the device. A person skilled in the art will appreciate that the housing can include various other features known in the art, and that the housing can have virtually any shape and size.

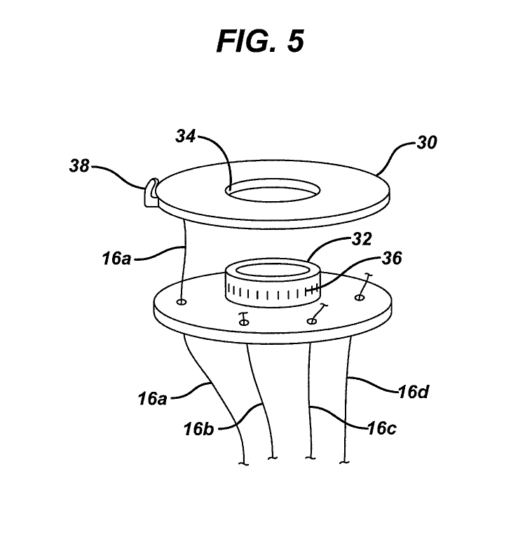

As indicated above, an actuator for moving the axial support 16 can be coupled to or formed on the cannula housing 27. The actuator can have a variety of configurations to facilitate movement of the axial support member 16, but in one exemplary embodiment the actuator, shown in FIG. 5, is adapted to rotate about an axis of the cannula 10 to slide the axial support member 16 along this axis. To achieve this rotation, in one embodiment the actuator can be in the form of a rotatable knob 30 which can rotate to gather the axial support member 16 around a spool 32 formed on the cannula housing 27. This pulls on the axial support member 16, causing the braided tube 12 to dilate from the first, undilated configuration to the second, dilated configuration. The amount of dilation of the braided tube 12 can vary depending on the amount of rotation of the rotatable knob 30 and the amount of the axial support member 16 that is gathered therearound. This allows the dilation of the braided tube 12, and thus the dilation of the tissue through which the braided tube 12 is disposed, to be controlled based on the amount of rotation of the rotatable knob 30.

The rotatable knob 30 can be rotatably coupled to the housing 27 and the axial support member 16 using various techniques. In the illustrated embodiment, the housing 27 includes four holes formed therein for receiving a proximal portion of four axial support members 16a, 16b, 16c, 16d therethrough. The holes in the housing 27 can be positioned in a variety of locations to allow the axial support members 16a-d to extend therethrough. In an exemplary embodiment, the holes are positioned radially outward from the braided tube 12 and from the spool 32 to allow the axial support members 16a-d to be pulled through the holes and wound around the spool 32. The proximal end of each axial support member 16a-d is fixedly mated to the rotatable knob 30, and the rotatable knob 30 includes an opening formed therethrough for receiving a portion of the spool 32. The knob 30 is thus rotatably coupled to the spool 32, and the proximal end of each axial support member 16a-d is attached to an outer portion of the knob 30. As the rotatable knob 30 is rotated, the axial support members 16a-d are pulled proximally and are gathered around the spool 32.

The rotatable knob 30 can also include features to allow the rotatable knob 30 to be maintained in a fixed position, thereby maintaining the braided tube 12 in a fixed dilated position. In one embodiment, the rotatable knob 30 can include a ratchet having a rack 36 and a pawl 34 to maintain a position of the rotatable knob 30. The pawl 34 can be adapted to engage a plurality of teeth formed on the rack 36, which can be formed on a portion of the rotatable knob 30. The pawl 34 will thus maintain the position of the rotatable knob 30 as it is being rotated around the spool 32. A person skilled in the art will appreciate that any mechanism can be used to maintain the position of the rotatable knob 30, including various locking mechanisms known in the art.

In order to release the axial support members 16a-d from the spool 32 to allow the braided tube 12 to return to the first, undilated configuration, the rotatable knob 30 can include a release mechanism 38 coupled thereto. The release mechanism 38 can be adapted to move the pawl 34 out of engagement from the rack 36 disposed on the spool 32. In one embodiment, the release mechanism 38 is movably associated with the pawl 34, and can be actuated to cause the pawl 34 to disengage from the teeth of the rack 36. Once disengage, the axial support members 16a-d can be unwound and released from the spool 32 to cause the length of the braided tube 12 to increase and the diameter of the braided tube 12 to decrease.

As previously indicated, the trocar assembly 20 can also include an obturator 22 that is removably disposable through the cannula 10 for introducing the cannula 10 through tissue. The obturator 22 can have a variety of configurations, and various obturators known in the art can be used, including rigid and flexible obturators for laparoscopic and endoscopic applications. In the illustrated embodiment, the obturator 22 has a hollow, elongate shaft with a proximal end that is coupled to a housing 25 and a distal end with a tip 28 that is adapted to be inserted through tissue. The tip 28 can have a variety of configurations to facilitate its insertion through tissue. For example, the tip 28 can be in the form of a tapered sharpened point to penetrate through tissue. Alternatively, or in addition, the tip 28 can include wings extending longitudinally along opposed sides thereof for separating tissue. The size of the elongate shaft of the obturator 22 can vary, but it preferably has a length that allows it to be inserted through the cannula 10 with the tip 28 extending beyond the distal end 14d of the braided tube 12.

The housing 25 formed on the proximal end of the elongate shaft can have a variety of configurations, but in an exemplary embodiment the housing 25 is provided to allow the obturator 22 to removably mate to a housing 27 on the cannula 10, as discussed above. For example, the housing 25 can include one or more mating elements to mate to corresponding mating elements on the housing 27 formed on the cannula 10. A release mechanism can be used to release the obturator 22 from the cannula 10. A person skilled in the other will appreciate that various techniques can be used to mate the housing 25 of the obturator 22 to the housing 27 of the cannula 10, including twist-lock mechanisms, threads, snap-fit, interference fit, etc. The obturator 22 also does not need to include a housing, but rather can merely be an elongate shaft that is slidably disposable through the trocar cannula 10.

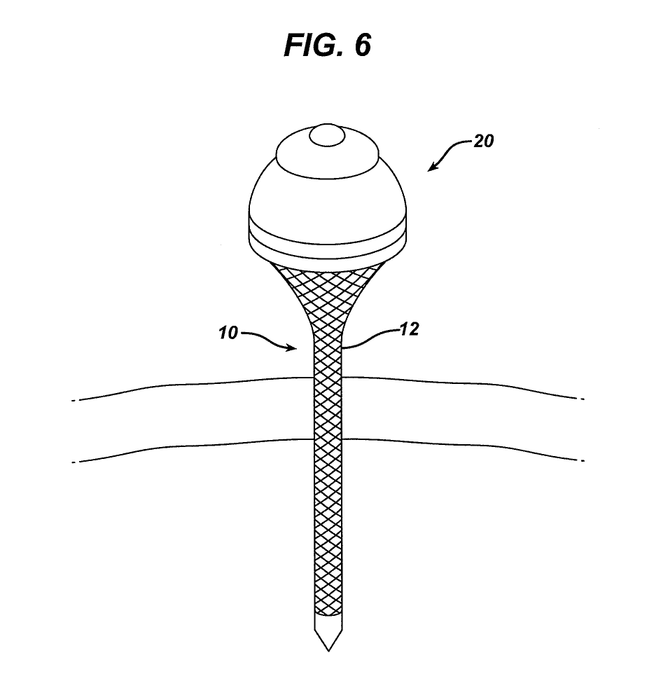

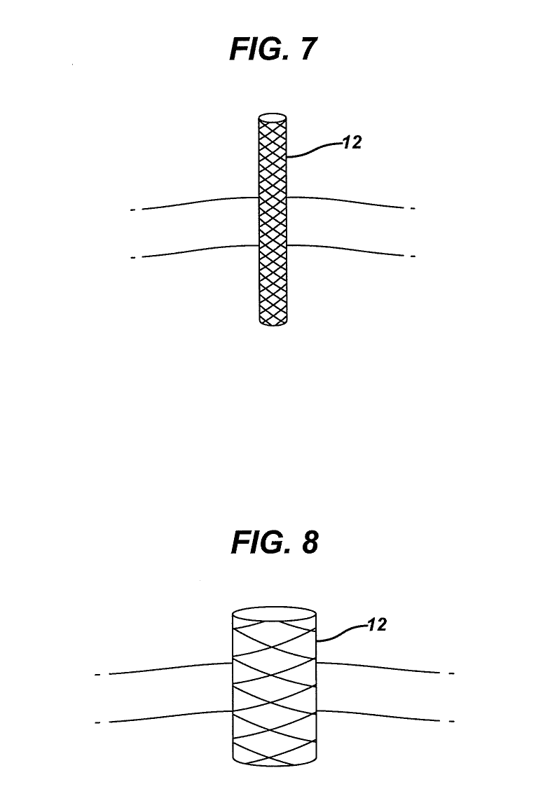

A method for dilating tissue is also provided herein. A person skilled in the art will appreciate that the braided tube 12 described herein can be used in any procedure, and that the trocar cannula assembly 20 is merely discussed as an example of a device that can utilize the braided tube 12. In one embodiment, the trocar cannula assembly 20 as described above can be inserted through tissue using the tip 28 of the obturator 22. In particular, a small incision can be made in the skin, and the trocar cannula assembly 20 can be inserted therethrough, as shown in FIG. 6, with the braided tube 12 of the cannula 10 in the first, undilated configuration. For example, the cannula 10 can be disposed through the abdominal wall, as shown in FIG. 7, and the braided tube 12 of the cannula 10 can be used to dilate tissue of the abdominal wall to allow access into the peritoneal cavity, as shown in FIG. 8. The proximal end 14p of the braided tube 12 can be just proximal of the outer wall of the tissue through which the cannula 10 has been inserted, and the distal end 14d of the braided tube 12 can be just distal of the inner wall of a cavity, such as the peritoneal cavity, through which the cannula 10 has been inserted to provide a low profile. The low profile of the braided tube 12 on both sides of the tissue opening, for example, through the abdomen, allows for a decrease in interference of access to the surgical field. The braided tube 12 of the cannula 10 can then be dilated to dilate the tissue therearound, either before or after the obturator 22 is removed from the cannula. The cannula 10 can be held in place, for example, by an interference fit with the tissue opening when the cannula 10 is dilated. In particular, a user can rotate the rotatable knob 30 to pull the axial support member 16 proximally and to wind the axial support member 16 around the spool 32 coupled to the rotatable knob 30. This causes the axial support member 16 to pull the distal end of the cannula 10 proximally, which in turn causes the length of the cannula 10 to decrease and the diameter of the cannula 10 to increase, thus dilating the cannula 10 and tissue disposed around the cannula 10. The axial support member 16 can provide rigidity to the braided tube 12 of the cannula 10 to prevent the braided tube 12 from collapsing after the obturator 22 is removed. To maintain the position of the rotatable knob 30 and the dilation of the cannula 10, the ratchet and pawl 34 can lock the rotatable knob 30 in a rotated position. When the procedure is complete, the release mechanism 38 can be released to release the axial support member 16 from the rotatable knob 30, causing the diameter of the cannula 10 to decrease and the length of the cannula 10 to increase. One advantage of dilating tissue using the braided tube 12 of the cannula 10 is that the incision through which the device is inserted can necessarily have a size or length that is significantly less than a diameter of the instruments inserted therethrough.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present invention.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A trocar cannula, comprising:
a braided tube having an inner lumen extending along a longitudinal axis between first and second ends; and
at least one axial support member slidably disposed along a length of the braided tube and adapted to move longitudinally to selectively increase and decrease a diameter of the inner lumen of the braided tube.

2. The cannula of claim 1, wherein the at least one axial support member extends from the distal end of the braided tube to a proximal end of the braided tube.

3. The cannula of claim 1, wherein the at least one axial support member extends from the distal end of the braided tube to a position distal to a proximal end of the braided tube.

4. The cannula of claim 1, wherein the at least one axial support member is coupled to an actuator adapted to slide the at least one axial support member longitudinally along the cannula.

5. The cannula of claim 4, wherein the at least one axial support member has a proximal end coupled to the actuator and a distal end coupled to a distal end of the braided tube.

6. The cannula of claim 4, wherein the actuator is adapted to pull the at least one axial support member proximally to increase the diameter of the inner lumen and decrease a length of the braided tube.

7. The cannula of claim 1, wherein the at least one axial support member is woven into the braided tube.

8. The cannula of claim 1, further comprising a plurality of axial support members spaced radially around the braided tube.

9. The cannula of claim 1, further comprising a flexible sheath disposed around an outer sidewall of the braided tube such that the sidewall is substantially fluid impermeable.

10. The cannula of claim 1, wherein the braided tube includes a coating formed thereon such that the braided tube is fluid impermeable.

11. The cannula of claim 1, further comprising one or more protrusions extending radially outward from the braided tube and adapted to engage the tissue.

12. A trocar, comprising:
an obturator; and
a cannula removably disposable within the obturator, the cannula being formed from a braided tube having at least one axial support member slidably disposed along a length thereof and adapted to move to vary a diameter of the cannula.

13. The trocar of claim 12, wherein the at least one axial support member is coupled to an actuator adapted to slide the at least one axial support member along the length of the cannula.

14. The trocar of claim 13, wherein the at least one axial support member includes a proximal end coupled to the actuator and a distal end coupled to a distal end of the cannula.

15. The trocar of claim 13, wherein the actuator is adapted to rotate about an axis of the cannula to slide the at least one axial support member along the axis.

16. The trocar of claim 13, wherein the actuator comprises a rotatable knob adapted to rotate to gather the at least one axial support member around a spool rotatably coupled to the rotatable knob to pull on the at least one axial support member.

17. The trocar of claim 16, further comprising a ratchet coupled to the rotatable knob and adapted to maintain the rotatable knob in a fixed position.

18. The trocar of claim 17, wherein the ratchet includes a release mechanism adapted to release the at least one axial support member to decrease the diameter of the cannula.

19. The trocar of claim 12, further comprising four axial support members spaced radially around the cannula.

20. A method of dilating tissue, comprising:
inserting a cannula into tissue;
pulling at least one axial support member slidably disposed along a length of the cannula to decease a length of the cannula and increase a diameter of the cannula and thereby dilate the tissue.

21. The method of claim 20, wherein the cannula is inserted into tissue with an obtruator disposed therein.

22. The method of claim 20, wherein pulling the at least one axial support member comprises rotating a rotatable knob coupled to the at least one axial support member such that the at least one axial support member is gathered around a spool formed on the rotatable knob.

23. The method of claim 22, wherein a ratchet maintains the rotatable knob in a fixed position to thereby maintain the at least one axial support member in a desired gathered position around the spool on the rotatable wheel.

24. The method of claim 23, further comprising releasing a release mechanism associated with the rotatable knob to release the at least one axial support member from the spool and decrease the diameter of the cannula.
